(19) United States
(12) Patent Application Publication
(10) Pub. No.: US 2016/0270749 A1
(43) Pub. Date: Sep. 22, 2016

(54) Methods for performing bi-planar fluoroscopy-guided surgical procedures

(71) Applicant: Ravish Patwardhan, New York City, NY (US)

(72) Inventor: Ravish Patwardhan, New York City, NY (US)

(21) Appl. No.: 15/077,853

(22) Filed: Mar. 22, 2016

Related U.S. Application Data
(60) Provisional application No. 62/136,620, filed on Mar. 22, 2015.

(51) Int. Cl. A61B 6/00

(52) U.S. Cl. CPC A61B 6/485; A61B 6/4441; A61B 6/463; A61B 6/4405; A61B 6/4411

(57) ABSTRACT

The present invention relates to methods and systems for bi-planar fluoroscopy, such as with dual fluoroscopes mounted to C-arms for lateral and anterior-posterior (AP) fluoroscopy of a patient. In one aspect of the invention, a bi-planar fluoroscopy system may generally include first and second fluoroscopes. In general, each fluoroscope may be positioned and aimed to provide different viewing angles and/or orientations of the target, such as, for example, placement for lateral and AP views of the target. In some exemplary embodiments, both fluoroscopes may be maintained as “fixed or locked” in AP and lateral positions, respectively, for example, and then each controlled by a set of proximally located control mechanisms, such as one control mechanism (e.g. a foot pedal or voice command specific to operation of a given fluoroscope each). The control mechanisms may further be adapted for use by a single operator individually and/or simultaneously. This may be particularly desirable for spinal procedures, such as for pedicle screw or kyphoplasty balloon placement, and may provide significantly enhanced efficiency and time reduction for these procedures, by completely negating subsequent movements after initial AP and lateral positions have been fixed with C-arms locked into such positions.

### CROSS-REFERENCE TO RELATED APPLICATIONS

This application claims the benefit and priority of U.S. provisional patent application Ser. No. 62/136,620, filed Mar. 22, 2015, entitled “Methods for performing bi-planar fluoroscopy-guided surgical procedures”, the contents of which is hereby incorporated by reference in its entirety.

### FIELD OF THE INVENTION

This invention relates generally to methods and systems for bi-planar fluoroscopy, particularly to systems of bi-planar fluoroscopes simultaneously controlled by a single operator and methods of using thereof. This invention further relates to methods of surgical intervention utilizing bi-planar fluoroscopes simultaneously controlled by a single operator, such as, for example, spinal procedures such as placement of pedicle screws and kyphoplasty.

### BACKGROUND OF THE INVENTION

It is often desirable to take X-rays of a patient from a number of different positions, preferably without the need for frequent repositioning of the patient or re-positioning of the C-arm(s). It is preferable that the X-ray support structure not unduly encumber the space immediately surrounding the patient to enable a physician to treat or otherwise attend to the patient without the need to repeatedly remove and replace the X-ray equipment. Mobile C-arm X-ray diagnostic equipment has been developed to meet these needs and has become well known in the medical art of surgical and other interventional procedures.

In pedicle screw placement procedures, initially, the patient is positioned on a frame on the operating room table in the prone position. In a typical procedure, a single C-arm mounted fluoroscope is utilized and the C-arm must be moved each time an image is taken, often about at least 20 times during the case, requiring sterility to be potentially compromised each time since to shoot an anterior-posterior (AP) image, one part of the C-arm goes under the operating table, which is a non-sterile environment. Once the operation starts, a sharp needle is placed at the lateral portion of the pedicle (which is verified using the AP position of the C-arm) and in the middle of the shaft of the pedicle (which is verified using the lateral position of the C-arm). This typically involves verbal instructions to the technician to move the C-arm from lateral to AP, then again to lateral, and so forth with each tap, and back and forth, each time re-sterilizing (using sterile drapes which are wasted), which may involve 5 or 6 taps at least to ensure one is entering the pedicle and not exiting the vertebral body in the anterior position (i.e. going too far anteriorly, which would be dangerous to rupture large vessels and abdominal structures). Once the needles are placed for percutaneous minimally invasive pedicle screw placement, the “K-wires” are placed through the cannulated needles, as place holders, and cannulated pedicle screws (since this is a minimally invasive pedicle screw system we are using), are placed. The series of AP and lateral C-arm motions must be used again, to ensure the screw is not “going too far”; finally, the rod is placed in a similar fashion, percutaneously for minimally invasive pedicle screw placement, ensuring on AP and lateral films that it is in the proper position.

In kyphoplasty, in a similar fashion as for pedicle screws, the pedicles are marked, and again needles are placed via the pedicles; however, since kyphoplasty is done for compression fractures, cement is released via these, and again AP and lateral views of C-arm are used for such placement to ensure the cement remains contained within the vertebral body as desired and does not enter into the disk space as avoidable, canal, or a vessel.

### SUMMARY OF THE INVENTION

The present invention relates to methods and systems for bi-planar fluoroscopy, such as with dual fluoroscopes mounted to C-arms for lateral and anterior-posterior (AP) fluoroscopy of a patient. The present invention more particularly relates to systems of bi-planar fluoroscopes simultaneously controlled by a single operator and methods of using thereof. The present invention further relates to methods of surgical intervention utilizing bi-planar fluoroscopes simultaneously controlled by a single operator, such as, for example, spinal procedures such as placement of pedicle screws and kyphoplasty, without requirement for verbal command or involvement of another operator or movement of the C-arm fluoroscope

In one aspect of the invention, a bi-planar fluoroscopy system may generally include first and second fluoroscopes, each of which may generally be mounted to an appropriate positioning device, such as a C-arm, for positioning and aiming a radiation source to pass radiation through a target, such as a patient or a part of a patient's body, and for the passed radiation to be received at a receiving device, such as a fluorescent screen, detector and/or sensor. In general, each fluoroscope may be positioned and aimed to provide different viewing angles and/or orientations of the target, such as, for example, placement for lateral and AP views of the target. The fluoroscopes may also include control mechanisms for triggering radiation exposure and/or image acquisition of the target, such as by a actuated control or triggering mechanism, which may be utilized by a user, such as a surgeon and/or other trained medical professional, during a medical procedure.

In some exemplary embodiments, both fluoroscopes may be controlled by a set of proximally located control mechanisms, such as one control mechanism for each fluoroscope. The control mechanisms may further be adapted for use by a single operator individually and/or simultaneously. In one embodiment, the control mechanisms may each include at least one foot pedal which may be pressed to trigger radiation exposure and/or image acquisition of the target. For example, the foot pedals may further be configured such that one foot pedal may be actuated by an operator's left foot and the other foot pedal may be actuated by the operator's right foot. In another example, a single foot pedal may be configured such that different motions of a single foot of the operator may trigger the first fluoroscope, the second fluoroscope, or both, such as, for further example, via a rocking motion from side to side, multiple control actuators on the pedal and/or any other appropriate configuration.

In another aspect of the present invention, a method for utilizing a bi-planar fluoroscopy system may include a single operator, such as surgeon and/or other trained medical professional, selectively actuating the control mechanisms for each fluoroscope during the course of a procedure to acquire the desired images of the target in real-time and/or quasi-real-time, such as to, for example, provide near immediate verification of placement, depth and/or trajectory of an implant, such as a pedicle screw or kyphoplasty balloon, during implantation. In some exemplary embodiments, the surgeon may utilize a dual fluoroscopy system, such as a system positioned to take both lateral and AP views, with dual foot pedals such that each pedal may be used to trigger image capture from one of the fluoroscopes at will by the surgeon without reliance on other personnel, without repositioning a fluoroscope and/or without occupying the surgeon's hands, and with dual displays for displaying captured images from both fluoroscopes simultaneously. Alternatively to the foot pedals, voice control may be utilized, such as with voice commands which may be recognized by the fluoroscopes to activate a specific image, e.g. in the AP or lateral orientation with respect to the patient's anatomy.

The present invention together with the above and other advantages may best be understood from the following detailed description of the embodiments of the invention and as illustrated in the drawings. The following description, while indicating various embodiments of the invention and numerous specific details thereof, is given by way of illustration and not of limitation. Many substitutions, modifications, additions or rearrangements may be made within the scope of the invention, and the invention includes all such substitutions, modifications, additions or rearrangements.

### BRIEF DESCRIPTION OF THE FIGURES

The drawings accompanying and forming part of this specification are included to depict certain aspects of the invention. A clearer impression of the invention, and of the components and operation of systems provided with the invention, will become more readily apparent by referring to the exemplary, and therefore non-limiting, embodiments illustrated in the drawings, wherein identical reference numerals designate the same components. Note that the features illustrated in the drawings are not necessarily drawn to scale.

FIGS. 1 and 1a illustrate embodiments of dual fluoroscopy systems;

FIGS. 2a, 2b and 2c illustrate views of a patient taken at lateral and AP views;

FIGS. 3 and 3a illustrate AP and lateral images taken in embodiments of the present invention; and

FIGS. 4, 4a, 4b, 4c, 4d and 4e illustrate the images taken during a typical single-C-arm method.

### DETAILED DESCRIPTION OF THE INVENTION

The detailed description set forth below is intended as a description of the presently exemplified methods, devices and compositions provided in accordance with aspects of the present invention, and is not intended to represent the only forms in which the present invention may be practiced or utilized. It is to be understood, however, that the same or equivalent functions and components may be accomplished by different embodiments that are also intended to be encompassed within the spirit and scope of the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the exemplified methods, devices and materials are now described.

The present invention relates to methods and systems for bi-planar fluoroscopy, such as with dual fluoroscopes mounted to C-arms for lateral and anterior-posterior (AP) fluoroscopy of a patient. The present invention more particularly relates to systems of bi-planar fluoroscopes simultaneously controlled by a single operator and methods of using thereof. The present invention further relates to methods of surgical intervention utilizing bi-planar fluoroscopes simultaneously controlled by a single operator, such as, for example, spinal procedures such as placement of pedicle screws and kyphoplasty.

In typical bi-planar fluoroscopy-guided procedures, a single fluoroscope is utilized to acquire images of the target by moving and/or orienting (and subsequently re-orienting) the fluoroscope to capture the appropriate image, such as, for example, by moving from lateral to AP positions about a patient. This method further generally requires additional personnel besides the surgeon and/or other medical professional performing the procedure to move, orient and/or trigger the fluoroscope, such as, for example, to maintain sterility and/or allow the surgeon to remain focused on and/or positioned properly for the procedure. The surgeon may further be required to give instructions frequently, such as when to move the fluoroscope, how to position it, when to trigger it, etc. This may often in sterility being lost, e.g. moving from the lateral to the AP position, and then re-establishment of sterility is required when returning to the lateral position with a single-C-arm method typically used. FIGS. 4, 4a, 4b, 4c, 4d and 4e illustrate the steps and images captured in a single C-arm method, with the steps in FIGS. 4d and 4e repeated about 3-4 times until the needle, such as a Jamshidi needle for example, is in the desired position. A total of about 10 verbal instructions to a technician and associated movement/reorientations of the C-arm may thus be utilized with a single C-arm for a single side of the patient, and about 20 for bilateral.

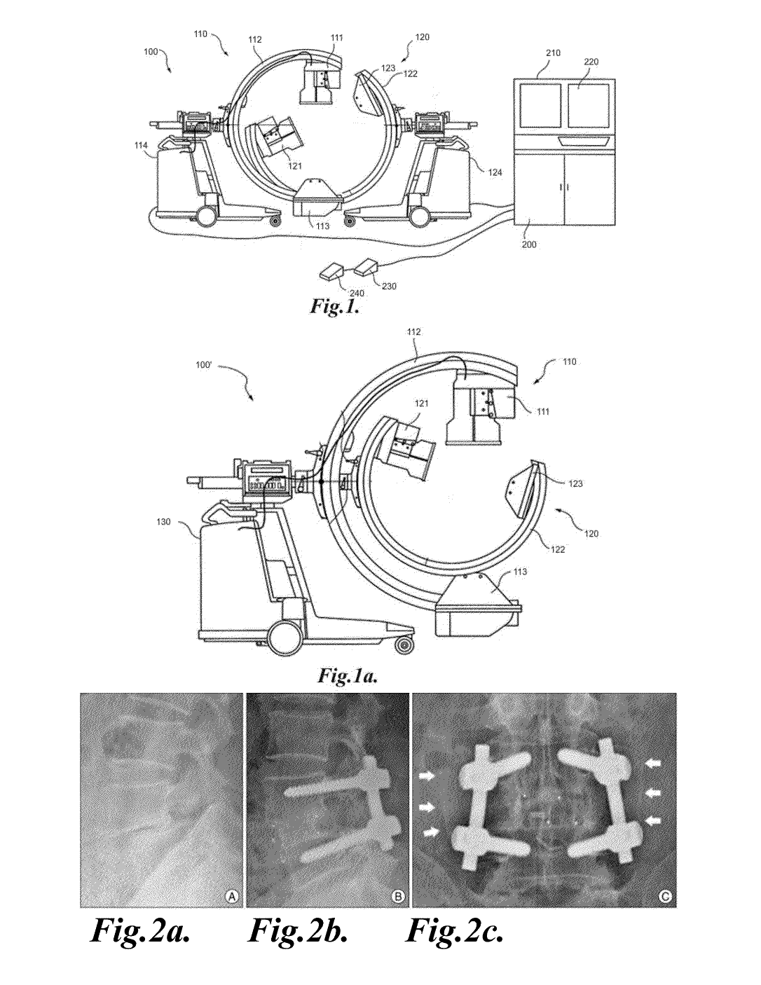

In one aspect of the invention, a bi-planar fluoroscopy system may generally include first and second fluoroscopes, each of which may generally be mounted to an appropriate positioning device, such as a C-arm, for positioning and aiming a radiation source to pass radiation through a target, such as a patient or a part of a patient's body, and for the passed radiation to be received at a receiving device, such as a fluorescent screen, detector and/or sensor. In general, each fluoroscope may be positioned and aimed to provide different viewing angles and/or orientations of the target, such as, for example, placement for lateral and AP views of the target. FIG. 1 illustrates an example of a dual fluoroscopy system 100 which includes a first fluoroscope 110 with a radiation source 111 and detector 113 mounted to a C-arm 112 and a second fluoroscope 120 with a radiation source 121 and a detector 123 mounted to a C-arm 122. In some embodiments, each fluoroscope 110, 120 may be on separate movable C-arms 112, 122, such as on separate carts 114, 124, as illustrated in FIG. 1. In other embodiments, the fluoroscopes 110, 120 may be mounted onto a single dual-C-arm mechanism 130, as illustrated in FIG. 1a.

Dual fluoroscopes may generally be desirable as it may eliminate the need for moving the fluoroscopes during a procedure in order to change views, such as with a single fluoroscope, and may thus save time, effort and/or remove the need for instructions to move the fluoroscope, which may further reduce the chance of mistakes and/or mishaps during a procedure and allow the surgeon to devote less attention to the fluoroscope operation. The fluoroscopes may also include control mechanisms for triggering radiation exposure and/or image acquisition of the target, such as by an actuated control or triggering mechanism, which may be utilized by a user, such as a surgeon and/or other trained medical professional, during a medical procedure.

In some exemplary embodiments, both fluoroscopes may be controlled by a set of proximally located control mechanisms, such as one control mechanism for each fluoroscope. The control mechanisms may further be adapted for use by a single operator individually and/or simultaneously. In one embodiment, the control mechanisms may each include at least one foot pedal which may be pressed to trigger radiation exposure and/or image acquisition of the target. Foot pedals may generally be desirable as they may allow the operator free use of the hands, which may further need to remain sterile during a procedure. Thus foot pedals may also reduce the need for re-sterilization of the hands during a procedure and/or the sterilization of the control mechanism. Further, foot pedals allow sterility to be maintained for the C-arm overall while being used. Alternatively, voice control may be utilized with voice commands specific for AP or lateral control of the C-arm.

For example, as illustrated in FIG. 1, the foot pedals may further be configured such that one foot pedal may be actuated by an operator's left foot and the other foot pedal may be actuated by the operator's right foot, such as with foot pedals 230, 240 as illustrated. In another example, a single foot pedal may be configured such that different motions of a single foot of the operator may trigger the first fluoroscope, the second fluoroscope, or both, such as, for further example, via a rocking motion from side to side, multiple control actuators on the pedal and/or any other appropriate configuration.

In another aspect of the present invention, a method for utilizing a bi-planar fluoroscopy system may include a single operator, such as surgeon and/or other trained medical professional, selectively actuating the control mechanisms for each fluoroscope during the course of a procedure to acquire the desired images of the target in real-time and/or quasi-real-time, such as to, for example, provide near immediate verification of placement, depth and/or trajectory of an implant, such as a pedicle screw or kyphoplasty balloon, during implantation.

In some exemplary embodiments, the surgeon may utilize a dual fluoroscopy system, such as a system positioned to take both lateral and AP views, with dual foot pedals such that each pedal may be used to trigger image capture from one of the fluoroscopes at will by the surgeon without reliance on other personnel, without repositioning a fluoroscope and/or without occupying the surgeon's hands, and with dual displays for displaying captured images from both fluoroscopes simultaneously. This may be particularly desirable for spinal procedures, such as for pedicle screw or kyphoplasty balloon placement, and may provide significantly enhanced efficiency and time reduction for these procedures. This configuration and usage may also be particularly desirable as it may maximize the usage of a surgeon's available dexterity (e.g. both hands and both feet performing tasks) without employing additional personnel.

In an exemplary embodiment, as illustrated with FIGS. 2a, 2b and 2c, a surgeon may capture simultaneous and/or near simultaneous images in a procedure, such as with the pedicle screw implantation as shown, utilizing a lateral fluoroscope to capture a lateral image in FIGS. 2a and 2b, and an AP fluoroscope to capture an AP image in FIG. 2c.

In some embodiments, a surgeon may capture simultaneous and/or near simultaneous images in a procedure, such as creating a tract for a wire or pedicle screw, for example, using a needle such as Jamshidi needle. In some embodiments, the fluoroscopes may be utilized to capture images that show the “streak” created in the images by the needle, which may generally form a streak on the image collinear with the needle and may thus indicate the trajectory of the needle's predicted path. This may be desirable to predict the path of the needle as it is being inserted or hammered into the target. This may be utilized in, for example, placement of posterior or anterior pedicle screws, such that the surgeon may verify a correct predicted path of the needle using the streak as a guide before beginning or continuing insertion so that the surgeon does not “miss” the desired target.

### Example of Placement of Pedicle Screws with Simultaneously Controlled Dual Fluoroscopes

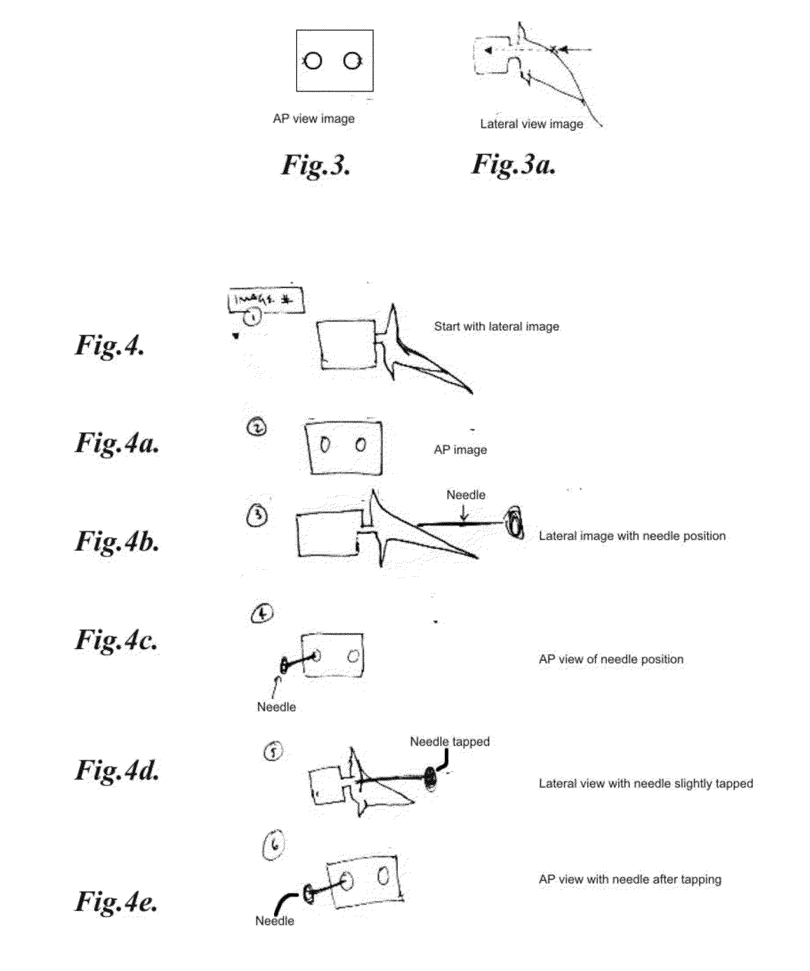

A surgeon-controlled (via foot-pedal, with typically the “on” button position for one C-arm under the left toes but not pressed since surgeon's weight is on his/her heels, while the right foot is similarly positioned over the other C-arm foot pedal but not depressed unless image is needed). Using this arrangement, the surgeon has both screens, e.g. displaying the AP and lateral fluoroscope views, in the surgeon's direct view, s/he can begin positioning for the pedicle access (e.g. for screw insertion or needle insertion into pedicle, for K-wire or kyphoplasty balloon placement). By real-time use by depressing the appropriate foot pedal needed, the surgeon may guide placement and view AP vs. lateral view without requiring (1) the frequent re-positioning that must constantly be undertaken when a single C-arm alternates AP and lateral views, such as, for example, after nearly every tap of the mallet, and requires repositioning by the radiology technician typically adding significant time and need for verbal commands; (2) constant further verbal direction to the radiology technician to “shoot an AP image” or “shoot a lateral image” and require the surgeon to designate which one while adding time and verbal commands while the surgeon could do so himself using a foot-pedal while saving both steps (1) and (2) since the C-arms are already positioned in AP and lateral views without need for verbal commands to move them or shoot the film to obtain the real-time image, expending a small fraction of the time and effort otherwise required. In this manner, for example, a surgeon could:
(1) identify the bony pedicles and mark entry sites on the skin prior to any skin incision, via a lateral pedicle point;
(2) after prepping and draping, make an incision projecting onto lateral border of pedicle (as visualized using surgeon-controlled foot pedal), then proceeding on a lateral-to-medial trajectory of insertion into a pedicle of a needle, for example (which may or may not be coupled to electronic sensors), and confirming position of depth again using surgeon-controlled foot pedals in real-time, remaining within the pedicle in both AP and lateral views as visualized again using surgeon-controlled foot pedals, negating constant need for AP and lateral repositioning as needed when a single C-arm is used or images must be voiced to technician to be taken and retaken constantly. Efficiency may be improved by over 90%, with only under 10% of the time taken compared to when this technique is not used, e.g. in insertion of pedicle screws or kyphoplasty balloons, with surgeon-controlled imaging allowing for greater safety by taking images as frequently as desired without repositioning C-arms and without requiring participation from another person (which could introduce greater error). In an optimal case, this may be accomplished with as little as 2 images, one AP and one lateral, as illustrated in FIGS. 3 and 3a, with the (x) marks showing starting points on the bones and the hash lines showing the trajectory of the needle.
(3) such steps could be carried out to completion of the procedure (e.g. percutaneous rod placement in pedicle screw cases to ensure rod seats in screws as desired, or cement placement to accomplish desired placement and avoid undesired migration of cement in kyphoplasty cases) by the surgeon control solely throughout using the foot pedals and maintaining sterility, while using his/her hands to operate, hence using all 4 limbs in a multi-dexterous fashion with resultant efficiency as noted previously.

Although the invention has been described with respect to specific embodiments thereof, these embodiments are merely illustrative, and not restrictive of the invention. The description herein of illustrated embodiments of the invention, including the description in the Abstract and Summary, is not intended to be exhaustive or to limit the invention to the precise forms disclosed herein (and in particular, the inclusion of any particular embodiment, feature or function within the Abstract or Summary is not intended to limit the scope of the invention to such embodiment, feature or function). Rather, the description is intended to describe illustrative embodiments, features and functions in order to provide a person of ordinary skill in the art context to understand the invention without limiting the invention to any particularly described embodiment, feature or function, including any such embodiment feature or function described in the Abstract or Summary. While specific embodiments of, and examples for, the invention are described herein for illustrative purposes only, various equivalent modifications are possible within the spirit and scope of the invention, as those skilled in the relevant art will recognize and appreciate. As indicated, these modifications may be made to the invention in light of the foregoing description of illustrated embodiments of the invention and are to be included within the spirit and scope of the invention. Thus, while the invention has been described herein with reference to particular embodiments thereof, a latitude of modification, various changes and substitutions are intended in the foregoing disclosures, and it will be appreciated that in some instances some features of embodiments of the invention will be employed without a corresponding use of other features without departing from the scope and spirit of the invention as set forth. Therefore, many modifications may be made to adapt a particular situation or material to the essential scope and spirit of the invention.

Reference throughout this specification to “one embodiment”, “an embodiment”, or “a specific embodiment” or similar terminology means that a particular feature, structure, or characteristic described in connection with the embodiment is included in at least one embodiment and may not necessarily be present in all embodiments. Thus, respective appearances of the phrases “in one embodiment”, “in an embodiment”, or “in a specific embodiment” or similar terminology in various places throughout this specification are not necessarily referring to the same embodiment. Furthermore, the particular features, structures, or characteristics of any particular embodiment may be combined in any suitable manner with one or more other embodiments. It is to be understood that other variations and modifications of the embodiments described and illustrated herein are possible in light of the teachings herein and are to be considered as part of the spirit and scope of the invention.

In the description herein, numerous specific details are provided, such as examples of components and/or methods, to provide a thorough understanding of embodiments of the invention. One skilled in the relevant art will recognize, however, that an embodiment may be able to be practiced without one or more of the specific details, or with other apparatus, systems, assemblies, methods, components, materials, parts, and/or the like. In other instances, well-known structures, components, systems, materials, or operations are not specifically shown or described in detail to avoid obscuring aspects of embodiments of the invention. While the invention may be illustrated by using a particular embodiment, this is not and does not limit the invention to any particular embodiment and a person of ordinary skill in the art will recognize that additional embodiments are readily understandable and are a part of this invention.

As used herein, the terms “comprises,” “comprising,” “includes,” “including,” “has,” “having,” or any other variation thereof, are intended to cover a non-exclusive inclusion. For example, a process, product, article, or apparatus that comprises a list of elements is not necessarily limited only those elements but may include other elements not expressly listed or inherent to such process, process, article, or apparatus.

1. A system for simultaneous bi-planar fluoroscopy by single operator comprising:
a first fluoroscope mounted to a first C-arm, said first fluoroscope outputting captured images to a first display;
a second fluoroscope mounted to a second C-arm, said second fluoroscope outputting captured images to a second display;
a first foot actuator coupled to said first fluoroscope, said first foot actuator being adapted to trigger image capture by said first fluoroscope when depressed; and
a second foot actuator coupled to said second fluoroscope, said second foot actuator being adapted to trigger image capture by said second fluoroscope when depressed;
wherein said first and second foot actuators are proximally located for operation by a single operator.

2. The system of claim 1 wherein said first fluoroscope is positioned and oriented to capture lateral images from a patient and said second fluoroscope is positioned and oriented to capture anterior-posterior (AP) images from said patient.

3. The system of claim 1, wherein said first and second C-arms are mounted on separate first and second carts, respectively.

4. The system of claim 1, wherein said first and second C-arms are mounted on a single cart.

5. A method for performing a spinal procedure on a patient comprising:
positioning a first and second fluoroscope for capturing images from a patient's spine from a first and second orientation, respectively, said first and second fluoroscopes each comprising a foot actuated image capture trigger proximal to a surgeon's feet; and
triggering at least one of said first and second fluoroscopes via said foot actuated image capture triggers during said spinal procedure with at least one of said surgeon's feet;
displaying acquired images from said at least one of said first and second fluoroscopes to said surgeon via a display.

6. The method of claim 5, further comprising placing a needle on a trajectory and triggering at least one of said first and second fluoroscopes via said foot actuated image capture triggers and acquiring a streak of said needle in said acquired images.

7. The method of claim 6, further comprising verifying the trajectory of said needle towards a desired target with said streak.

8. The method of claim 5, wherein said first orientation comprises a lateral orientation and said second orientation comprises an AP orientation.
